(19) United States
(12) Patent Application Publication
(10) Pub. No.: US 2012/0195988 A1
(43) Pub. Date: Aug. 2, 2012

(54) Therapeutic Uses for Plants of the Capsicum Genus

(76) Inventor: Ali Rezai-Fard, Hayes, Middlesex (GB)

(21) Appl. No.: 13/386,180

(22) PCT Filed: Jul. 20, 2010
(86) PCT No.: PCT/GB2010/001377

§ 371 Date: Feb. 10, 2012

(30) Foreign Application Priority Data
Jul. 20, 2009 (GB) 0912587.3

(51) Int. Cl. A61K 36/81; C07J 17/00; C07J 51/00; C07G 11/00; A61K 31/704; A61K 31/7028; A61K 31/56; A61P 31/10; A61P 35/00; A61P 39/02; A61P 31/00; A61P 31/04; A61P 31/14; A61P 31/12; A61P 31/18; A61K 48/00; C07G 3/00

(52) U.S. Cl. 424/760; 536/4.1; 536/5; 552/502; 514/26; 514/25; 514/169

(57) ABSTRACT

A saponin or a sapogenin, or a saponin or sapogenin derivative, from a plant of the capsicum genus for use in the treatment of a disease other than eczema and dermatitis. A process for preparing a plant of the capsicum genus, extracts, or seeds thereof for use as a medicament suitable for the treatment of diseases other than skin conditions which process comprises storing the plant, extracts or seeds thereof at a temperature of 20° C. or below.

### BACKGROUND

The present invention relates to the field of the therapeutic uses of certain compounds, particularly of the saponin and sapogenin type, and particularly to those saponins and sapogenins extracted from plants of the capsicum genus.

The compounds saponins and sapogenins are naturally occurring molecules found in a great variety of different plant species. A saponin is a type of glycoside and comprises a sugar component referred to as a glycone and a non-sugar component referred to as an aglycone. The two main types of saponins are steroidal saponins with a steroidal aglycone and triterpenoid saponins with a triterpenoid aglycone. A sapogenin is the aglycone component of the saponin, and these also occur on their own in nature without a glycone component.

Saponins and sapogenins have received interest because of their potential biological activities and therapeutic properties. They are also a component of many Chinese herbal remedies. For example, saponins and sapogenins found in plants of the plant families Liliaceae, Convallariaceae, Dioscoreaceae, Agavaceae, Smilacaceae and Amaryllidaceae are reported as having antifungal properties in Yang, C et al. Antimicrobial agents and chemotherapy, Vol. 50, p. 1710-1714. Saponins and sapogenins have also been shown to have anti-cancer properties such as the triterpenoid sapogenin Ursolic acid and its derivatives which are found to naturally occur in many different plants such as apples, bilberries, basil and cranberries. Other saponins have also been shown to exhibit in vitro cytotoxic activity against cancer cells, such as in Mskhiladze, L et al. Molecules 2008, 13, 2925-2934. Other uses of saponins and sapogenins include as adjuvants, antiviral and antiretroviral compounds such as in Ryu, S et al. Arch. Pharm. Res. 4, 339-342., and as anti-bacterial and anti-microbial agents such as in Soetan. K et al. African Journal of Biotechnology, 5, p2405-2407. It has also been speculated that sapogenins could be useful in gene therapy coupled to a gene when it is introduced to the body. A sapogenin with known anti-inflammatory properties could help reduce the immune response of the body to the new gene.

Currently, there remains a need to find new saponins and sapogenins with potential therapeutic properties and biological activity since not all potential sources of saponins and sapogenins have been explored, and exploited for therapeutic use.

Whilst saponins and sapogenins from different plant sources share a similar basic structure, the compounds from any particular source will be unique to that source and have their own specific structure. The properties of saponins and sapogenins isolated from one source will not, therefore, necessarily be shared by the saponins and sapogenins isolated from another source. Indeed, they could have different properties. Accordingly, the properties of the compounds from a particular source cannot readily be predicted.

A number of saponins and sapogenins can be isolated from the capsicum genus, and reference can be made to the applicant's published British patent application GB245585 for further details. However, these compounds have not to date been well investigated. The applicant now believes that, surprisingly, these compounds posses a number of further interesting properties.

### SUMMARY OF THE INVENTION

In a broad aspect of the invention, there is provided a saponin or sapogenin, or a saponin or sapogenin derivative, from a plant of the capsicum genus for use in the treatment of disease other than eczema and dermatitis.

In another aspect of the invention, there is provided a saponin or sapogenin, or a saponin or sapogenin derivative, from a plant of the capsicum genus for use in the treatment of disease other than eczema and dermatitis.

In one embodiment of the invention, there is provided a saponin or sapogenin, or a saponin or sapogenin derivative, from a plant of the capsicum genus for use as an anti-fungal agent, excluding the saponin CAY-1 from cayenne pepper (Capsicum frutescens).

In a second embodiment of the invention, there is provided a saponin or sapogenin, or a saponin or sapogenin derivative, from a plant of the capsicum genus for use as an anti-cancer agent, or for use as an agent used to treat the side affects of other cancer treatments.

In a third embodiment of the invention, there is provided a saponin or sapogenin, or a saponin or sapogenin derivative, from a plant of the capsicum genus for use as an adjuvant.

In a fourth embodiment of the invention, there is provided a saponin or sapogenin, or a saponin or sapogenin derivative, from a plant of the capsicum genus for use as an antiviral agent or an anti-retroviral agent.

In a fifth embodiment of the invention, there is provided a saponin or sapogenin, or a saponin or sapogenin derivative, from a plant of the capsicum genus for use as an anti-bacterial/anti-microbial agent.

In a sixth embodiment of the invention, there is provided a saponin or sapogenin, or a saponin or sapogenin derivative, from a plant of the capsicum genus for use in gene therapy.

In a seventh embodiment of the invention, there is provided a saponin or sapogenin, or a saponin or sapogenin derivative, from a plant of the genus capsicum for use in the alleviation of the symptoms of HIV infection or AIDS, or in the curing of HIV infection or AIDS.

In another aspect of the invention, there is provided a process for preparing a plant of the capsicum genus, extracts, or seeds thereof for use as a medicament suitable for the treatment of diseases other than skin conditions which process comprises storing the plant, extracts or seeds thereof at a temperature of 20° C. or below. In preferable embodiments, the diseases for treatment are the diseases referred to in preceding paragraphs, for treatment with saponins, sapogenins, and their derivatives, in accordance with the present invention.

Preferably, the above mentioned process is used to prepare a medicament suitable for the treatment of HIV infection or AIDS, or in the curing of HIV infection or AIDS.

In a highly preferred aspect, the saponin or sapogenin, or a derivative thereof, is from the seeds of a plant of the capsicum genus. That is, the seeds themselves are used. They are preferably washed to remove capsaicin.

Extracts from plants of the capsicum genus have been used therapeutically to treat various diseases and medical conditions. However, the prior art identifies the active ingredient of these capsicum plant extracts to be the molecule capsaicin, and teaches that it is capsaicin that causes capsicum plants to possess the therapeutic properties. Capsaicin is a pungent molecule that is responsible for the hot flavour of many plants of the capsicum genus. It also causes a burning sensation. If this molecule were to be used in the manufacture of a medicament for the treatment of disease, there would be obvious drawbacks to its usage due to the extremely pungent flavour and burning sensation it may cause to mucous membranes of a patient.

Saponins and sapogenins from the capsicum genus are described in the applicant's previous patent application, published as GB 245585. The applicant found evidence that the saponin and sapogenin compounds extracted from these plants of the capsicum genus could be active ingredients useful in the treatment of eczema and dermatitis.

The applicant has since found that these compounds could potentially be used in the treatment of a wide variety.of. other diseases. and conditions. The saponins and sapogenins that are the active ingredients of the present invention are from capsicum plants, and are specific to the capsicum genus. They are known as capsicidins or capsicidines. This includes the group of compounds known as the capsicosides A-G.

Use of saponins and sapogenins extracted from plants of the capsicum genus presents an improvement over prior art use of capsicum extracts which all contain the pungent, burning molecule capsaicin. Therapeutic use of these compounds for the conditions described therefore demonstrates a clear improvement over prior art medical uses of capsicum plant extracts containing capsaicin. Use of these saponins and sapogenins in the manufacture of medicaments in the treatment of disease other than eczema and dermatitis is disclosed for the first time in this application.

### DESCRIPTION OF PREFERRED EMBODIMENTS

The saponins or sapogenins useful in the present invention are from plants of the capsicum genus. The capsicum plants can be any of but are not limited to C. annuum, C. frutescens, C. chinense, C. pendulum, C. pubescens, C. minimum, C. baccatum, C. abbreviatum, C. anomalum, C. breviflorum, C. buforum, C. brasilianum, C. campylopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. frutescens, C. galapagoensis, C. geminifolum, C. hookerianum, C. lanceolatum, C. luteum, C. microcarpum, C. minutiflorum, C. mirabile, C. parvifolium, C. praetermissum, C. schottianum, C. scolnikianum, C. stramonifolium, C. tetraginum, C. tovarii, C. villosum, C. violaceum and their hybrids.

The compounds may be isolated from the plant using chemical means, or one or more parts of the plant may be used “whole”—for example, the seeds. The relevant part (e.g. seeds) may be used as they are or treated further—for example used in crushed or ground form.

The medicament can, for example, be a medicament comprising a pharmaceutical formulation that can be administered by any means, including but not limited to oral, nasal, rectal and other transmucosal forms of administration, topical, and intravenous forms of drug delivery. The medicament may also comprise one or more pharmaceutically acceptable excipients. Preferably, the saponins and sapogenins for use in the context of the invention are administered to the human or animal body topically, orally, intranasally or systemically.

In the context of the present invention, a saponin is a naturally occurring glycoside that comprises a sugar component (glycone), and a non sugar component (aglycone). In the context of this invention, the saponin can be derived from any plant of the capsicum genus, and examples are given above. The glycone component of the saponin may be any sugar unit, or any chain of any length of any sugar units wherein each sugar unit is linked to the next sugar unit in the chain by a glycosidic bond.

The aglycone component of the saponin may be any fused polycyclic ring system. Preferably, the aglycone component is a carbocyclic steroidal ring system or a polycyclic triterpenoid ring system. A steroidal unit may be referred to as a tetracyclic triterpene, and a triterpenoid unit may be referred to as a pentacyclic triterpene. The saponins useful in the present invention can have any of the above described structures, provided said saponins can be found in or extracted from plants of the capsicum genus. In a most preferred embodiment the aglycone component of the saponin is a triterpenoid unit.

In the context of the present invention, a sapogenin is a naturally occurring compound that can be derived from any plant from the capsicum genus. A sapogenin is an aglycone component of a saponin that exists in nature on its own and without the glycone component of the saponin. In the context of this invention, a sapogenin may be any polycyclic ring system. Preferably, the sapogenin is a steroidal unit or a triterpenoid unit. In the context of this invention the steroidal units may be referred to as tetracyclic triterpenes, and the triterpenoid units may be known as pentacyclic triterpenes. In a most preferred embodiment, the sapogenin is a triterpenoid unit.

Both the aglycones of the saponins, and the sapogenins of the present invention may comprise heterocyclic polycyclic ring systems, such as the alkaloid functional group. The polycyclic ring systems of the saponin aglycones and sapogenins may also have any number or type of functional group bonded to any of the carbon atoms in the polycyclic ring system. The bonding configurations between the carbon atoms in the polycyclic ring systems may also be of any oxidation state and any level of saturation. In this way, the subject matter of the invention also comprises those saponins and sapogenins that have an array of unsaturated carbon-carbon double bonds in the polycyclic ring system, any organic functional group attached to the ring systems and even any heteroatom such as but not limited to nitrogen, oxygen or sulphur present within the carbon based polycyclic triterpene ring framework.

With reference to the preceding paragraph and to the statements of invention, the term saponin or sapogenin derivative is taken to comprise any derivative or type of compound related to the capsicum derived saponins or sapogenins. This encompasses but is not limited to the alkaloids, ketals, esters and amines of the saponins/sapogenins of the present invention. This also includes the various subclasses of steroidal saponins and sapogenins such as spirostanol, furostanol, pregnane, solasodane, and solanidane type steroidal saponins/sapogenins.

Saponins and sapogenins useful in the present invention can have any of the structures or structural features described in the preceding paragraphs, provided that the compounds can be found in or extracted from plants of the capsicum genus, and that they are not the saponin known in the art as CAY-1.

A saponin or sapogenin with anti-fungal activity may, for example, be used to treat the symptoms or causes of any fungal infection in the human or animal body. The fungal infection may be a topical fungal infection such as athlete's foot or systemic fungal infections.

A saponin or sapogenin as described herein can be used directly as a chemotherapeutic agent in the treatment of tumours and cancer in the human or animal body. The saponins and sapogenins may be used to shrink tumours and kill cancer cells as part of chemotherapy. They may also decrease the swelling caused by cancers. The saponins/sapogenins may also be used to alleviate many of the side effects and symptoms caused by cancer and by various cancer treatments such as chemotherapy and radiation therapy. These include but are not limited to reducing nausea that is a common side affect in chemo/radiotherapy, reducing allergic reactions that might occur before transfusions and to other chemotherapeutic drugs, lessen the headaches commonly caused by brain tumours, and to increase appetite. Saponins and sapogenins from the capsicum genus, along with their derivatives, may be used to treat any form of cancer including but not limited to breast, liver, lung, prostate, testicular, ovarian, cervical, uterus, brain, skin, colon, throat, mouth, bone, pancreas, stomach and lymph node cancer.

Within the context of the invention, an adjuvant is considered to be any agent that modifies the effect of other agents like drugs and vaccines. A saponin/sapogenin from the capsicum genus may be a pharmaceutic adjuvant, a hormonal adjuvant, an immunological adjuvant, or any other type of adjuvant for therapeutic use with the human body.

A saponin or sapogenin from the capsicum genus may be used as an antiviral or anti-retroviral agent. Examples include, but are not limited to, antiviral agents that are viral entry inhibitors, viral uncoating inhibitors, agents that interfere with viral synthesis, viral protease inhibitors, agents that act at the assembly and release phase of a virus, or agents that work by interfering with viruses at any stage in their life cycle.

A saponin or sapogenin from the capsicum genus may be used in accordance with the present invention in the treatment of HIV infection, or AIDS (acquired immune deficiency syndrome). In some embodiments, the treatment can be to alleviate the symptoms of HIV or AIDS. In other embodiments, the saponins/sapogenins for use in accordance with the present invention can be used in the curing of HIV or AIDS while in still other embodiments the saponins/sapogenins for use in accordance with the present invention can be used to delay the onset of HIV infection, or stop an HIV infection developing into AIDS.

Without being limited by theory, it is believed that the anti-HIV properties of the saponins/sapogenins for use in accordance with the present invention arise because the saponins/sapogenins work by a mechanism that inhibits maturation of the HIV virus. It is believed that this mechanism of operation is similar to that of the antiretroviral compound berivimat, a derivative of the natural product betulinic acid. This is that the compound is thought to bind to an HIV polyprotein precursor that forms part of the capsid (protein coat of the virus). This causes defects in the capsid and prevents the virus binding to other cells, and infecting them.

A saponin or sapogenin, or a saponin or sapogenin derivative, from a plant of the capsicum genus may be used as an anti-bacterial/anti-microbial agent. Examples of their use include, but are not limited to natural antibiotics, synthetic antibiotics, antifungals, antiparasitics or as any other antimicrobial agents.

Saponins or sapogenins, or a saponin or sapogenin derivative, from a plant of the capsicum genus may also be used in gene therapy. They may be used in any form of gene therapy. The saponins or sapogenins of the invention may, for example, be coupled with a gene before the gene is introduced to the human or animal body for therapeutic purposes. As the gene is antigenic to the host it is being delivered to, the host will produce an inflammatory immune response to the gene. If the gene is coupled to a saponin or sapogenin of the present invention, the saponin or sapogenin may have an anti-inflammatory affect to the host and hence aid in the successful acceptance of the new gene into the patient.

The saponins and sapogenins for use in the present invention can be from or extracted from any plant of the capsicum genus. In a preferred embodiment, the saponins and sapogenins are from plants of the species C. annuum or C. frutescens, particularly green cayenne chilli peppers. In a highly preferred embodiment, the saponins and sapogenins are from plants of the C. annuum or C. frutescens species, particularly green or red bird's eye chillis or thin Indian chilli peppers.

The saponins and sapogenins for use in the present invention can be from or extracted from any part of the capsicum plant, including but not limited to the flesh of the fruit, the seeds, the membranes holding the seeds to the flesh, the stalks, the leaves, the roots or any constituent, derivative or extract obtained therefrom. In a preferred embodiment, the saponins and sapogenins are extracted from the seeds of capsicum plants. The saponins and sapogenins may be extracted from the seeds of the capsicum plants by any suitable method.

The saponins or sapogenins for use in the present invention can also be extracted from any synthetic version of any constituent, derivative or extract obtained from plants or parts of plants of the capsicum genus.

In an embodiment, the capsicum seeds are first removed and separated from the flesh of the capsicum plant. The capsicum seeds may be removed from the fruit of the capsicum plant by any suitable method. The capsicum seeds may, for example, be removed manually from the flesh of the capsicum plant.

The capsicum seeds may be washed, for example with a liquid such as water to remove the chemical capsaicin that might contaminate the surface of the capsicum seeds. Any suitable liquid may be used, but water is preferred. The water that washes the capsicum seeds may be any temperature, but preferably from 20° C. to 70° C. Most preferably, the water that washes the capsicum seeds is “hot to the touch” at approximately 50° C. Preferably, the washing is carried out repeatedly to remove as much capsaicin as possible from the seeds. The seeds can be checked qualitatively for the presence of capsaicin by tasting.

The amount of saponin or sapogenin to be ingested for treatment of the disease types listed in the preceding paragraphs should be an amount suitable to give a pharmacological effect. For example, the amount may be derived from the amount present in the range of from approximately 0.1 seeds to 30 seeds per kg of body weight per day, for example from about 1 seed to about 20 seeds per kg of body weight per day.

In one embodiment, the amount of saponin or sapogenin to be ingested orally for treatment of the disease types listed in the preceding paragraphs can be standardised to the amount of saponin or sapogenin that can be derived from approximately 400 to 500 capsicum seeds. This dosage of saponin or sapogenin can, for example, be ingested twice a day for successful treatment.

It is probable that the beneficial effects of saponins or sapogenins extracted from the capsicum plants in treating the disease types referred to in the preceding paragraphs are maximised if the source from which they are derived, (for example chilli peppers or their seeds) are stored in a cool environment before use.

The source (for example chilli peppers or their seeds) is preferably stored before use in a cool environment of 20° C. or below, or 15° C. or below, for example between −10° C. and 20° C., preferably from 0° C. to 10° C., most preferably from 0° C. to 5° C., and most preferably from 2° C. to 4° C.

It is thought that the therapeutic properties of the saponins or sapogenins from capsicum plants are maximised if the plants or their seeds are stored in a cool environment for at least 2 days prior to use. It is thought that the therapeutic properties of the saponins or sapogenins could be a direct result of being stored in a cool environment for a period of time. This time period could be any period of time long enough to cause the therapeutic. effects of the saponins or sapogenins to be suitably enhanced and made suitable for therapeutic use. Preferably, this time period is of up to 8 weeks, for example from 1 to 5 weeks, or from 1 to 3 weeks. In a highly preferred embodiment, the time period of storage in a cool environment is for around 8 weeks. However, in some embodiments, the time period for storage may be only a few days.

Based on the inventor's investigations to date, it appears that the saponins or sapogenins have the greatest therapeutic effect in treatment of diseases referred to in the preceding paragraphs when the capsicum seeds/plants they are extracted from are stored at from 0 to 10° C. for up to about 8 weeks, for example from 1 to 3 weeks.

With reference to the preceding paragraphs, the duration of storage of the seeds/extracts of or whole capsicum plants at the low temperatures described above is believed to affect the dose of saponin/sapogenin required to have a pharmacological effect. It is believed that the longer the saponins or sapogenins are stored at low temperature for, the lower the dose of plant material (e.g. seeds) required to have the desired curative pharmacological effect. Without being limited by theory, it is believed that the process of storing the saponins/sapogenins extracted from capsicum plants, seeds of capsicum plants, extracts of capsicum plants, or whole capsicum plants at temperatures within the ranges described in preceding paragraphs causes a build up of the saponin/sapogenin active ingredient within the capsicum plants/plant extracts/seeds. Therefore, a longer period of storage as described above, within the temperature ranges as described above will cause more of the saponin/sapogenin active ingredient to be present in the capsicum plants, extracts or seeds. In using the saponins/sapogenins in the treatment of disease in accordance with the present invention, the amount of saponin/sapogenin required to have a therapeutic or curative effect, for example, will be the amount derived from a certain quantity of seeds. The necessary quantity of seeds will be less if the seeds have been stored within the temperature ranges as described above for a longer period of time. This is because the saponin/sapogenin active ingredient. will. have had time to build up to a greater quantity over the duration of storage at the low temperatures.

Therefore, the quantity of the saponin/sapogenin active ingredient is believed to be linked to storage at low temperature, and to duration of storage time.

For example, although not claimed as part of the invention, the active ingredient of the extracts of capsicum seeds for treating skin conditions described in GB 0800791.6 is believed by the applicant to be the saponins/sapogenins for use in accordance with the present invention. The applicant found that when capsicum seeds were stored at a temperature of from 2 to 4° C. for around three weeks, the amount of active ingredient required to treat eczema was the amount found in around 500 seeds, to be taken twice daily for around 21 days. It is believed that if the capsicum seeds had been stored for longer periods of time at this temperature, the dose would be less.

The applicant believes that the saponin/sapogenin active ingredient located within the capsicum plants, most likely in the seeds of the capsicum plants, may be what is referred to as a phytoalexin. The term phytoalexin as used herein refers to any chemical produced by any part of a plant as part of its defence in response to an “elicitor” (or trigger or stress). The elicitors can be biotic (living) or abiotic (non-living). Phytoalexins of different plant species are chemically diverse with different types being characteristic of particular plant species. Without limiting, examples of phytoalexins include terpenoids, glycosteroids and alkaloids. Phytoalexins can be antimicrobial. They may work by mechanisms such as cell wall puncturing, delaying microbial maturation, disrupting metabolism, or preventing reproduction. The applicant believes that the saponins/sapogenins for use in accordance with the present invention could be phytoalexins of the capsicum plant, and in particular a phytoalexin produced due to the abiotic elicitor of cold temperature. This would explain why storage of capsicum plants at the low temperatures described above for the periods of time as described above could lead to a build up of the saponin/sapogenin active ingredient in the capsicum plants. It would also explain why increased storage time at low temperature would mean the therapeutic dose of saponin/sapogenin required would be derived from a smaller amount of capsicum seeds.

It is possible that other compounds found in plants of the capsicum genus in addition to saponins and sapogenins may be partly responsible for the therapeutic and beneficial effects. These include, but are not limited to, 24-®-ethyl-lophenol, 24-methylene-cycloartanol, 24-methyl-lanost-8-en-3-β-ol, 31-nor-lanost-9(11)-en-3-β-ol, 31-nor-lanosterol, 4-α-14-α-24-trimethyl-cholesta-8(24)-dien-3-β-ol, β-amyrin, citrostadienol, cycloartanol, cycloartenol, cycloeucalenol, gramisterol, lanost-8-en-3-β-ol, lanosterol, lophenol, lupeol and obtusifoliol. Accordingly, one or more of these compounds may be used either instead of, or in combination with, one or more saponin or sapogenins.

1. A medicament comprising saponin or a sapogenin, or a saponin or sapogenin derivative, from a plant of the capsicum genus for use in the treatment of a disease other than eczema and dermatitis.

2. The medicament according to claim 1, wherein the disease is a disease other than a skin condition.

3. The medicament according to claim 1, for use as an anti-fungal agent, and wherein the saponin or sapogenin, or saponin or sapogenin derivative for use is not the saponin CAY-1 from cayenne pepper (Capsicum frutescens).

4. The medicament according to claim 1, for use as an anti-cancer agent, or for use as an agent to treat the side affects of other cancer treatments.

5. The medicament according to claim 1, for use as an adjuvant, antiviral agent, antiretroviral agent, anti-bacterial/anti microbial agent.

6. The medicament according to claim 1, for use in the treatment of HIV infection/AIDS, or in the alleviation of symptoms of HIV infection/AIDS.

7. The medicament according to claim 1, for use in gene therapy.

8. The medicament according to claim 1, wherein the saponin or sapogenin, or a derivative thereof is not used in combination with the compound capsaicin.

9. The medicament according to claim 1, wherein the saponin or sapogenin, or derivatives thereof, are the capsicidins or capsicidines, or the group of compounds known as capsicosides A to G.

10. The medicament according to claim 1, wherein the plants of the capsicum genus are selected from one or more of C. annuum, C. frutescens, C. chinense, C. pendulum, C. pubescens, C. minimum, C. baccatum, C. abbreviatum, C. anomalum, C. breviflorum, C. buforum, C. brasilianum, C. campylopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. frutescens, C. galapagoensis, C. geminifolum, C. hookerianum, C. lanceolatum, C. luteum, C. microcarpum, C. minutiflorum, C. mirabile, C. parvifolium, C. praetermissum, C. schottianum, C. scolnikianum, C. stramonifolium, C. tetraginum, C. tovarii, C. villosum, C. violaceum and their hybrids.

11. The medicament according to claim 1, wherein the saponin, sapogenin or derivative thereof is suitable for administration by oral, nasal, rectal, topical, intravenous, or transmucosal administration.

12. The medicament according to claim 1, wherein the saponin or sapogenin, or derivatives thereof comprise a steroidal unit or a triterpenoid unit.

13. The medicament according to claim 1, wherein the saponin or sapogenin derivative comprises an alkaloid, ketal, ester or amine.

14. The medicament according to claim 5, wherein the saponin/sapogenin or derivative thereof is a viral entry inhibitor, viral uncoating inhibitor, agent that interferes with viral synthesis, viral protease inhibitor, or an agent that acts at the assembly and release phase of a virus.

15. The medicament according to claim 14, wherein the saponins or sapogenins, or derivatives thereof inhibit maturation of the HIV virus.

16. The medicament according to claim 1, wherein the seeds of the plant are used.

17. The medicament according to claim 1, wherein the active ingredient(s) is extracted from the plant.

18. The medicament according to claim 16, wherein the seeds are washed before use.

19. The medicament according to claim 18, wherein the seeds are washed with liquid, preferably water.

20. The medicament according to claim 18, wherein washing of the seeds is done at a temperature of from 20° C. to 70° C.

21. The medicament according to any of claims 16, wherein the seeds are stored before use at a temperature of below 20° C.

22. The medicament according to any of claims 16, wherein the seeds are stored before use for a time period of up to eight weeks.

23. The medicament according to claim 1, the saponin, sapogenin, or derivative thereof is a phytoalexin specific to a plant of the genus capsicum.

24. A process for preparing a plant of the capsicum genus, extracts, or seeds thereof for use as a medicament suitable for the treatment of diseases other than skin conditions which process comprises storing the plant, extracts or seeds thereof at a temperature of 20° C. or below.

25. A process according to claim 24, wherein the plant of the capsicum genus, extracts, or seeds thereof is stored at a temperature of 20° C. or below for a time period of up to 8 weeks before said plants, extracts or seeds thereof are used in the manufacture of a medicament.

26. A process according to claim 24, wherein the plant, extracts or seeds thereof is stored at a temperature of from −10° C. to 20° C., or from 0° C. to 10° C. or from 0° C. to 5° C.

27. A process according to claim 24, wherein the plant, extracts or seeds thereof is stored for a time period of 4 to 8 weeks.

28. A process according to claim 24, wherein the plant, extracts or seeds thereof is stored for a time period of between 1 and 3 weeks.

29. A process according to claim 24, wherein the medicament is suitable for use the treatment of a disease other than eczema and dermatitis.

30. A process according to claim 24, wherein the medicament is free of capsaicin.

31. A process according to claim 24, wherein the medicament is suitable for administration by oral, nasal, rectal, topical, intravenous, or transmucosal administration.

32. A process according to claim 24, wherein the plant of the capsicum genus is any of the plant are selected from one or more of C. annuum, C. frutescens, C. chinense, C. pendulum, C. pubescens, C. minimum, C. baccatum, C. abbreviatum, C. anomalum, C. breviflorum, C. buforum, C. brasilianum, C. campvlopodium, C. cardenasii, C. chacoense, C. ciliare, C. ciliatum, C. chlorocladium, C. coccineum, C. cordiforme, C. cornutum, C. dimorphum, C. dusenii, C. exile, C. eximium, C. fasciculatum, C. fastigiatum, C. flexuosum, C. frutescens, C. galapagoensis, C. geminifolum, C. hookerianum, C. lanceolatum, C. luteum, C. microcarpum, C. minutiflorum, C. mirabile, C. parvifolium, C. praetermissum, C. schottianum, C. scolnikianum, C. stramonifolium, C. tetraginum, C. tovarii, C. villosum, C. violaceum and their hybrids.

33. A process according to claim 24, wherein the medicament is suitable for use as a viral entry inhibitor, viral uncoating inhibitor, agent that interferes with viral synthesis, viral protease inhibitor, or an agent that acts at the assembly and release phase of a virus.

34. A process according to claim 24, wherein the medicament functions by inhibiting maturation of the HIV virus.

35. A pharmaceutical composition comprising a saponin or a sapogenin or derivative thereof of claim 1 and a pharmaceutically acceptable carrier.

36. The medicament according to claim 6, wherein the saponin/sapogenin or derivative thereof is a viral entry inhibitor, viral uncoating inhibitor, agent that interferes with viral synthesis, viral protease inhibitor, or an agent that acts at the assembly and release phase of a virus.

37. The medicament according to claim 36, wherein the saponins or sapogenins, or derivatives thereof inhibit maturation of the HIV virus.

38. The medicament according to claim 19, wherein washing of the seeds is done at a temperature of from 20° C. to 70° C.

39. The medicament according claim 18, wherein the seeds are stored before use at a temperature of below 20° C.

40. The medicament according to claim 19, wherein the seeds are stored before use at a temperature of below 20° C.

41. The medicament according to claim 20, wherein the seeds are stored before use at a temperature of below 20° C.

42. The medicament according to claim 18, wherein the seeds are stored before use for a time period of up to eight weeks.

43. The medicament according to claim 19, wherein the seeds are stored before use for a time period of up to eight weeks.

44. The medicament according to claim 20, wherein the seeds are stored before use for a time period of up to eight weeks.
